(19) United States
(12) Patent Application Publication
(10) Pub. No.: US 2008/0051492 A1
(43) Pub. Date: Feb. 28, 2008

(54) Inverse Emulsions as Thickeners for Cosmetics

(75) Inventors: Andrea Mitarotonda, Bray (IE); Arianna Benetti, Gallarate (VA) (IT); Giuseppe Li Bassi, Gavirate (VA) (IT)

Correspondence Address: PAUL S MADAN;MADAN, MOSSMAN & SRIRAM, PC, 2603 AUGUSTA DRIVE, SUITE 700, HOUSTON, TX, 77057-5662, US

(73) Assignee: LAMBERTI SpA, Albizzate (VA) (IT)

(21) Appl. No.: 11/832,402

(22) Filed: Aug. 1, 2007

(30) Foreign Application Priority Data
Aug. 23, 2006 (IT) IT-VA2006A000049

(51) Int. Cl. C08J 3/00; A61K 8/06

(52) U.S. Cl. 523/336; 424/401; 514/772.3

(57) ABSTRACT

Inverse emulsions, useful in applications such as cosmetics, are those wherein the weight ratio between the aqueous phase and the organic phase is from 4:1 to 2:1 and containing from 20 to 70% by weight of an acrylic polymer comprising monomeric units deriving from: i) 2-acrylamido-2-methylpropanesulfonic acid; ii) at least one cationic acrylic monomer of formula I:

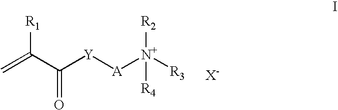

wherein R₁ is hydrogen or methyl; R₂, R₃, R₄ are, one independently of the others, hydrogen or C₁-C₄ alkyl; Y is NH or O; A is a C₁-C₆ alkylene; X is an anion; iii) acrylic or methacrylic acid; iv) at least one polyfunctional monomer, the inverse emulsion being characterized by the fact that in the polymer the monomeric units deriving from 2-acrylamido-2-methylpropanesulfonic acid represent from 1% to 29% molar of the total monomers i), ii) and iii). It is emphasized that this abstract is provided to comply with the rules requiring an abstract which will allow a searcher or other reader to quickly ascertain the subject matter of the technical disclosure. It is submitted with the understanding that it will not be used to interpret or limit the scope or meaning of the claims.

### CROSS REFERENCE TO RELATED APPLICATION

This Application Claims Priority from Italian Patent Application having the serial number IT VA2006A000049 which was filed on Aug. 3, 2006.

### BACKGROUND OF THE INVENTION

1. Field of the Invention

The present invention relates to inverse emulsions useful as thickeners in cosmetic formulations and to the procedure for their preparation.

2. Background of the Art

It is known that a technical problem often encountered in the cosmetic industry is to obtain high viscous formulations such as pastes and gels that are stable over time and exhibit high compatibility with skin and hair.

Methods of preparing high viscous formulation include those wherein an inverse emulsion containing a polymer comprising units deriving from acrylamide, 2-acrylamido-2-methylpropanesulfonic acid and a polyfunctional monomer are used. A disadvantage of such inverse emulsions is the fact that they contain traces of acrylamide, a toxic substance which is unacceptable by the present European legislative trend.

Another approach is one wherein cross-linked or branched polyelectrolytes based on strongly acidic monomers and other monomers, but not acrylamide, are used. In this approach, an inverse emulsion is described.

In another approach, copolymers of carboxylic acids and quaternary ammonium compounds are used in the preparation of gels and emulsions.

Another approach is found to include the use of copolymers of a neutral monomer (N-alkylacrylamide) with one or more monomers selected among cationic monomers, monomers bearing strongly acidic functional groups and monomers bearing weakly acidic functional groups.

In yet another approach, inverse emulsions comprising a polymer obtained by polymerization of two or more anionic acrylic monomers and of at least one cationic monomer is known. In this approach, at least one of the anionic acrylic monomers includes a strongly acid functionality and specifically a sulfonic functional group.

### SUMMARY OF THE INVENTION

In one embodiment, the invention is an inverse emulsion comprising: an aqueous phase, and an organic phase wherein: the weight ratio of the aqueous phase to the organic phase is from 4:1 to 2:1; the inverse emulsion includes from 20 to 70% by weight of an acrylic polymer; the acrylic polymer is prepared by polymerizing: i) 2-acrylamido-2-methylpropanesulfonic acid; ii) at least one cationic acrylic monomer of formula I:

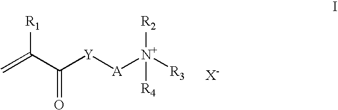

wherein R₁ is: hydrogen or methyl; R₂, R₃, R₄ are, one independently of the others, hydrogen or C₁-C₄ alkyl; Y is NH or O; A is a C₁-C₆ alkylene; and X is an anion; iii) acrylic or methacrylic acid; and iv) at least one polyfunctional monomer; and wherein from 1 molar % to 29 molar % of the total molar % of monomers i), ii) and iii) used to prepare the polymer is 2-acrylamido-2-methylpropanesulfonic acid.

In another embodiment, the invention is a method for the preparation of an inverse emulsion for cosmetic formulations comprising: a. preparing a mixture comprising from 40 to 60% by weight of a water phase, and from 60 to 40% by weight of a mixture of acrylic monomers consisting of: i) 2-acrylamido-2-methylpropanesulfonic acid; ii) at least one cationic acrylic monomer of formula I:

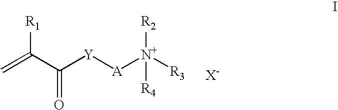

wherein R₁ is: hydrogen or methyl; R₂, R₃, R₄ are, one independently of the others, hydrogen or C₁-C₄ alkyl; Y is NH or O; A is a C₁-C₆ alkylene; and X is an anion; iii) acrylic or methacrylic acid; and wherein from 1 molar % to 29 molar % of the total molar % of monomers i), ii) and iii) is 2-acrylamido-2-methylpropanesulfonic acid; b. adding to the composition an alkali to regulate the pH between 4 and 7; c. adding to the composition prepared in b from 0.1 to 10 mmoles of a polyfunctional monomer per mole of mixture of monomers i), ii) and iii) and an initiator of radical polymerization while maintaining the temperature between 3 and 7° C.; d. preparing an organic phase containing one or more water-in-oil emulsifiers; e. introducing the mixture obtained in b. into the organic phase prepared in c. and emulsifying the two phases by stirring; f. initiating the polymerisation and completing it while maintaining the temperature at from 55 to 95° C. under stirring; and g. cooling the reaction mixture to 35-45° C. and adding thereto an oil-in-water emulsifier.

### DETAILED DESCRIPTION OF THE INVENTION

The inverse emulsions of the present invention comprise a crosslinked polymer obtained by polymerisation of: acrylic and/or methacrylic acid, 2-acrylamido-2-methylpropanesulfonic acid and a cationic monomer.

The inverse emulsions of the invention beside possessing high skin and hair compatibility and exhibiting good thickening properties and stability over time, show high thickening power not only in aqueous solutions, but also in oil and water emulsions; these properties makes them particularly suited for the preparation of cosmetic formulations.

With the expression “cosmetic formulations” we mean the products normally used for personal care, such as body and face creams, hair gels and lotions, hair colouring and bleaching creams, sunscreen compositions, make-up products, cleansing, moisturizing and perspiring fluids and other products for similar applications.

By cosmetic product with high compatibility with skin and hair we mean a product that is easily absorbed through a keratinous substrate while making changes in the touch, in moisturization and perspiration, and improving the general sensorial characteristics without altering the physiological pH.

A further essential characteristic of the thickeners employed in cosmetic formulations is that they manifest their thickening capability without negatively altering the other properties of the formulations. It is moreover highly desirable in the cosmetic field to have thickeners in the form of stable emulsion that are able to give stable cosmetic formulations.

With the expression “stable emulsion” we mean an emulsion that in the normal storing conditions (from −10° C. to 40° C.) and for the usual lifetime (180-360 days) does not show phase separation, sediment, formation of floating pellicles and lumps.

With the expression “stable cosmetic product” we mean a cosmetic formulation that in the above said conditions and lifetime does not show phase separation, sediment, formation of floating pellicles and lumps.

In the specialised literature many methods are reported to regulate the rheological properties of different formulations, often including the use of polymers in the form of inverse emulsion (an inverse emulsion is an emulsion containing both an oil-in-water emulsifier and a water-in-oil emulsifier, wherein the aqueous phase is dispersed in the organic phase in very small drops), but the synthetic thickeners for cosmetics of the present invention are never described. It is a fundamental object of the present invention to prepare an inverse emulsion useful for the preparation of cosmetic formulations wherein the weight ratio between the aqueous phase and the organic phase is from 4:1 to 2:1 and containing from 20 to 70% by weight of an acrylic polymer comprising monomeric units deriving from:
i) 2-acrylamido-2-methylpropanesulfonic acid;
ii) at least one cationic acrylic monomer of formula I:

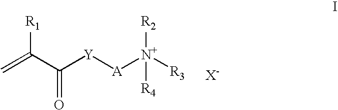

wherein R₁ is hydrogen or methyl; R₂, R₃, R₄ are, one independently of the others, hydrogen or C₁-C₄ alkyl; Y is NH or O; A is a C₁-C₆ alkylene; X is an anion;
iii) acrylic or methacrylic acid; and
iv) at least one polyfunctional monomer. The inverse emulsion is characterized by the fact that in the polymer, the monomeric units deriving from 2-acrylamido-2-methylpropanesulfonic acid represent from 1% to 29% molar of the total monomers i), ii) and iii).

The present invention also includes a procedure for the preparation of an inverse emulsion for cosmetic formulations characterised by:
a. preparing a composition consisting of from 40 to 60% by weight of water, and for the remaining percentage by weight of a mixture of acrylic monomers consisting of i) 2-acrylamido-2-methylpropanesulfonic acid in acid form, or partially or totally in the form of salt; ii) at least one cationic acrylic monomer of formula I:

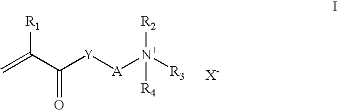

wherein R₁ is hydrogen or methyl; R₂, R₃, R₄ are, one independently of the others, hydrogen or C₁-C₄ alkyl; Y is NH or O; A is a C₁-C₆ alkylene; X is an anion; iii) acrylic or methacrylic acid. The mixture is further characterized by the fact that 2-acrylamido-2-methylpropanesulfonic acid represents from 1% to 29% molar of the total monomers i), ii) and iii);
b. adding to the mixture an alkali to regulate the pH between 4 and 7;
c. adding to the mixture prepared in b. from 0.1 to 10 mmol of a polyfunctional monomer per mole of mixture of monomers i), ii) and iii) and an initiator of radical polymerisation, maintaining the temperature between 3 and 7° C.;
d. preparing an organic phase containing one or more water-in-oil emulsifiers;
e. introducing the mixture obtained in b. into the organic phase prepared in c. and emulsifying the two phases by vigorous stirring;
f. initiating the polymerisation and completing it maintaining the temperature between 55 and 95° C. under vigorous stirring; and
g. cooling the reaction mixture to 35-45° C. and adding an oil-in-water emulsifier.

In the general formulas for the cationic acrylic monomers, the X is defined as an anion. This counter ion may be any that will not cause the cationic acrylic monomer to be insoluble or otherwise prevent it from being polymerized. For example, X can be a halide such as a chloride or bromide. While the general formula appears to require the anion to have a −1 charge, it is not so limited. For example, it may be a complex or simple anion having a −2 charge subject to the prior stated requirement that the counter ion not prevent the cationic acrylic monomer from being polymerized.

The cationic acrylic monomer of formula I is present in the acrylic polymer of the invention in a molar percentage comprised between 0.1 and 10% on the total sum of monomers i), ii) and iii) and is preferably chosen from acryloyloxyethyl-trimethylammonium chloride and methacryloyloxyethyl-trimethylammonium chloride.

The polyfunctional monomer contains two or more unsaturated reactive groups and it is present in the acrylic polymer in an amount comprised between 0.01 and 1 mmoles per mole of mixture of monomers i), ii) and iii). In some embodiments, the polyfunctional monomer is methylenebisacrylamide.

In the procedure according to the invention it is preferred to use the sodium salt of 2-acrylamido-2-methylpropanesulfonic acid, and to regulate the pH of phase b. with aqueous sodium hydroxide (NaOH).

Among the initiators of radical polymerisation useful for with the present invention are ammonium, potassium or sodium persulfate, and water-soluble organic peroxides. For example hydrogen peroxide and peracetic acid may be so used.

In the inverse emulsions of the invention the organic phase consists of mineral oils containing saturated hydrocarbons or by vegetable oils or by mixture thereof having boiling point from 150 to 300° C. Preferably the organic phase is mineral oil, polydecene, isohexadecane, or a C₁₃-C₁₆ isoparaffin; more preferably it is C₁₃-C₁₆ isoparaffin.

The water-in-oil and the oil-in-water emulsifiers are those normally used for this purpose. Useful water-in-oil emulsifiers include: sorbitan monolaurate, sorbitan monopalmitate, sorbitan monostearate, and sorbitan monooleate. Useful oil-in-water emulsifiers include the linear or branched ethoxylated alcohols. The total amount of emulsifiers in the inverse emulsion of the invention is from 2 to 10% by weight; the ratio between water-in-oil emulsifiers and oil-in-water emulsifiers may range between 2:1 and 1:2.

To initiate the polymerization of the acrylic monomers an aqueous solution of sodium bisulfite may advantageously be used.

The inverse emulsions of the invention may further additionally contain the common additives used in radical polymerisation, by way of example sequestering agents such as sodium diethylenetriaminepentaacetate.

As it was previously observed, the inverse emulsions of the present invention are particularly suited for the treatment of hair and skin, in body and face creams, hair gels and lotions, hair colouring and bleaching creams, sunscreen compositions, make-up products, cleansing, moisturizing and perspiring fluids.

In the following examples the preparation of inverse emulsions according to the invention and of an inverse emulsion according to the prior art, prepared with % of 2-acrylamido-2-methylpropanesulfonic acid higher than 29% (according to WO 2004/113393), are reported. The application tests show the very high thickening power of the emulsions of the present invention, in aqueous solution, in glycols, and in emulsified water and oil systems.

The stability of water and oil emulsion systems comprising the inverse emulsions of the invention was also evaluated; the stability is correlated to the stability of the cosmetic products containing the emulsions of the invention as thickeners.

The examples illustrate the present invention without limiting it, nor the kind of application of the inverse emulsions of the invention.

### EXAMPLE 1

The following ingredients are loaded into a 1.5 l Pyrex reactor equipped with a steel anchor stirrer:
45.5 g of de-mineralized water;
445.5 g of an 50% by weight aqueous solution of sodium 2-acrylamido-2-methylpropane sulfonate (0.972 mol);
179.9 g (2.499 mol) of acrylic acid; and
0.78 g (0.003 mol) of ADAMQUAT MC 80 (acryloyloxyethyl-trimethyl ammonium chloride sold by Atofina).

After a cooling down period, necessary to reach a temperature close to 0° C., the following ingredient are slowly added while stirring:
149.8 g of a 50% aqueous NaOH solution
10 g aqueous solution (1% by weight) of methylenebisacrylamide;
0.5 g of a solution (40% by weight) of sodium diethylenetriaminepentaacetate; and
11.9 g of a solution (4% by weight) of ammonium persulfate.

In the meantime, the organic phase is prepared inside a 500 ml beaker adding under stirring:
20 g of sorbitan monooleate; and
232.5 g of C₁₃-C₂₀ isoparaffin

The aqueous phase is slowly added into the organic phase and subsequently the mixture is efficiently stirred with a high shear dispersing machine (ULTRA-TURRAX IKA). The emulsion obtained is then reloaded in the reactor and the reaction is ready to be started (reaction phase). The first operation is to insufflate nitrogen directly in the bulk of the product for about 10 minutes. This is a key step, because it enables to lower and control the amount of oxygen dissolved in the emulsion and to adjust the induction times. The second phase takes place only after the emulsion temperature is warmed up to 20° C. After that, 23.9 g of a 1% by weight aqueous solution of sodium metabisulfite is quickly loaded drop-wise through an addition funnel. The third phase is the radical reaction. The reaction proceeds spontaneously raising gradually the temperature to about 60° C. in 50 minutes. The stirring is maintained very fast and cool water re-circulates inside the reactor jacket. After this period of time the emulsion is heated to 60° C. and maintained at this temperature for about one hour to complete the monomers conversion, consuming the residual monomers. Subsequently a cooling down period is required to reach a temperature of 35-40° C. The final step is the addition of 25 g of C₁₂-C₁₆ linear alcohol 8 moles ethoxylated.

The mixture is rapidly stirred till homogeneity is reached; the final emulsion (Emulsion 1) is then unloaded and stored for at least 24 hours before the evaluation of its properties.

### EXAMPLE 2

The following ingredients are loaded into a 1.5 l Pyrex reactor equipped with a steel anchor stirrer:
45.5 g of de-mineralized water;
445.5 g of an 50% by weight aqueous solution of sodium 2-acrylamido-2-methylpropane sulfonate (0.972 mol);
179.9 g (2.499 mol) of acrylic acid; and
0.78 g (0.003 mol) of ADAMQUAT MC 80 (acryloyloxyethyl-trimethyl ammonium chloride sold by Atofina).

After a cooling down period, necessary to reach a temperature close to 0° C., the following ingredient are slowly added while stirring:
149.8 g of a 50% aqueous NaOH solution
10 g aqueous solution (1% by weight) of methylenebisacrylamide;
0.5 g of a solution (40% by weight) of sodium diethylenetriaminepentaacetate; and
11.9 g of a solution (4% by weight) of ammonium persulfate.

In the meantime, the organic phase is prepared inside a 500 ml beaker adding under stirring:
20 g of sorbitan monooleate; and
232.5 g of C₁₆ isoparaffin

The aqueous phase is slowly added into the organic phase and subsequently the mixture is efficiently stirred with a high shear dispersing machine (ULTRA-TURRAX IKA). The emulsion obtained is then reloaded in the reactor and the reaction is ready to be started (reaction phase). The first operation is to insufflate nitrogen directly in the bulk of the product for about 10 minutes. This is a key step, because it enables to lower and control the amount of oxygen dissolved in the emulsion and to adjust the induction times. The second phase takes place only after the emulsion temperature is warmed up to 20° C. After that, 23.9 g of a 1% by weight aqueous solution of sodium metabisulfite is quickly loaded drop-wise through an addition funnel. The third phase is the radical reaction. The reaction proceeds spontaneously raising gradually the temperature to about 60° C. in 50 minutes. The stirring is maintained very fast and cool water re-circulates inside the reactor jacket. After this period of time the emulsion is heated to 60° C. and maintained at this temperature for about one hour to complete the monomers conversion, consuming the residual monomers. Subsequently a cooling down period is required to reach a temperature of 35-40° C. The final step is the addition of 25 g of C₁₂-C₁₆ linear alcohol 8 moles ethoxylated.

The mixture is rapidly stirred till homogeneity is reached; the final emulsion (Emulsion 2) is then unloaded and stored for at least 24 hours before the evaluation of its properties.

### EXAMPLE 3

The following ingredients are loaded into a 1.5 l Pyrex reactor equipped with a steel anchor stirrer:
45.5 g of de-mineralized water;
445.5 g of an 50% by weight aqueous solution of sodium 2-acrylamido-2-methylpropane sulfonate (0.972 mol);
179.9 g (2.499 mol) of acrylic acid; and
0.78 g (0.003 mol) of ADAMQUAT MC 80 (acryloyloxyethyl-trimethyl ammonium chloride sold by Atofina).

After a cooling down period, necessary to reach a temperature close to 0° C., the following ingredient are slowly added while stirring:
149.8 g of a 50% aqueous NaOH solution
10 g aqueous solution (1% by weight) of methylenebisacrylamide;
0.5 g of a solution (40% by weight) of sodium diethylenetriaminepentaacetate; and
11.9 g of a solution (4% by weight) of ammonium persulfate.

In the meantime, the organic phase is prepared inside a 500 ml beaker adding under stirring:
20 g of sorbitan monooleate; and
232.5 g of C₂₀ hydrogenated polydecene

The aqueous phase is slowly added into the organic phase and subsequently the mixture is efficiently stirred with a high shear dispersing machine (ULTRA-TURRAX IKA). The emulsion obtained is then reloaded in the reactor and the reaction is ready to be started (reaction phase). The first operation is to insufflate nitrogen directly in the bulk of the product for about 10 minutes. This is a key step, because it enables to lower and control the amount of oxygen dissolved in the emulsion and to adjust the induction times. The second phase takes place only after the emulsion temperature is warmed up to 20° C. After that, 23.9 g of a 1% by weight aqueous solution of sodium metabisulfite is quickly loaded drop-wise through an addition funnel. The third phase is the radical reaction. The reaction proceeds spontaneously raising gradually the temperature to about 60° C. in 50 minutes. The stirring is maintained very fast and cool water re-circulates inside the reactor jacket. After this period of time the emulsion is heated to 60° C. and maintained at this temperature for about one hour to complete the monomers conversion, consuming the residual monomers. Subsequently a cooling down period is required to reach a temperature of 35-40° C. The final step is the addition of 25 g of C₁₂-C₁₆ linear alcohol 8 moles ethoxylated.

The mixture is rapidly stirred till homogeneity is reached; the final emulsion (Emulsion 3) is then unloaded and stored for at least 24 hours before the evaluation of its properties.

### COMPARATIVE EXAMPLE 4

The following ingredients are loaded into a 1.5 l Pyrex reactor equipped with a steel anchor stirrer:
62.21 g of de-mineralized water;
573 g of an 50% by weight aqueous solution of sodium 2-acrylamido-2-methylpropane sulfonate (0.972 mol);
135 g (2.499 mol) of acrylic acid; and
0.53 g (0.003 mol) of ADAMQUAT MC 80 (acryloyloxyethyl-trimethyl ammonium chloride sold by Atofina).

After a cooling down period, necessary to reach a temperature close to 0° C., the following ingredient are slowly added while stirring:
112.38 g of a 50% aqueous NaOH solution
10 g aqueous solution (1% by weight) of methylenebisacrylamide;
0.5 g of a solution (40% by weight) of sodium diethylenetriaminepentaacetate; and 10.75 g of a solution (4% by weight) of ammonium persulfate.

In the meantime, the organic phase is prepared inside a 500 ml beaker adding under stirring:
20 g of sorbitan monooleate; and
214.8 g of C₁₃-C₂₀ isoparaffin

The aqueous phase is slowly added into the organic phase and subsequently the mixture is efficiently stirred with a high shear dispersing machine (ULTRA-TURRAX IKA). The emulsion obtained is then reloaded in the reactor and the reaction is ready to be started (reaction phase). The first operation is to insufflate nitrogen directly in the bulk of the product for about 10 minutes. This is a key step, because it enables to lower and control the amount of oxygen dissolved in the emulsion and to adjust the induction times. The second phase takes place only after the emulsion temperature is warmed up to 20° C. After that, 21.5 g of a 1% by weight aqueous solution of sodium metabisulfite is quickly loaded drop-wise through an addition funnel. The third phase is the radical reaction. The reaction proceeds spontaneously raising gradually the temperature to about 60° C. in 50 minutes. The stirring is maintained very fast and cool water re-circulates inside the reactor jacket. After this period of time the emulsion is heated to 60° C. and maintained at this temperature for about one hour to complete the monomers conversion, consuming the residual monomers. Subsequently a cooling down period is required to reach a temperature of 35-40° C. The final step is the addition of 25 g of C₁₂-C₁₆ linear alcohol 8 moles ethoxylated.

The mixture is rapidly stirred till homogeneity is reached; the final emulsion (Emulsion 4) is then unloaded and stored for at least 24 hours before the evaluation of its properties.

### Property evaluation of Emulsions 1-4

5 oil/water emulsions are prepared with each of the Emulsions 1 and 4 (columns E1 and E4 in the table), having the following composition:

| | |
|---|---|
| Water | 78% |
| Oil phase | 20% |
| Emulsion 1 or 4 | 2% |

where the oil phase is respectively C12-C15 alkyl benzoate, mineral oil, cyclopentasiloxane, sunflower oil and ethylhexyl palmitate. The stability of the formulation is evaluated with the separation test in the centrifuge, at 25° C. and 6000 rpm. In the following table (Table 1) the percentages of separation after centrifugation are shown.

TABLE 1

Phase
separation
%

| Oil phase: | E1 | E4* |
|---|---|---|
| C12-C15 alkyl benzoate | 0 | 0 |
| Ethylhexyl palmitate | 1 | 1 |
| Sunflower oil | 0 | 0 |
| Mineral oil | 3 | 3 |
| cyclopentasiloxane | 0 | 0 |

*comparative

The thickening properties of the Emulsions 1 and 4 are instead evaluated by measuring their Brookfield viscosity at 2% in water, 25° C. and 5 rpm as well as the viscosity of the formulations of Table 1, in the same conditions. The obtained results are reported in Table 2.

TABLE 2

Viscosity
mPa * s

| Oil phase: | E1 | E4* |
|---|---|---|
| none (aqueous solution) | 52080 | 29840 |
| C12-C15 alkyl benzoate | 88000 | 56480 |
| Ethylhexyl palmitate | 68000 | 60960 |
| Sunflower oil | 80600 | 56640 |
| Mineral oil | 55040 | 85000 |
| cyclopentasiloxane | 56800 | 88000 |

*comparative

The thickening properties of the Emulsions 1 and 4 are also evaluated in glycerine and monopropylene glycol by measuring their Brookfield viscosity at 2%, 25° C. and 5 rpm in the said solvents. The results are reported in Table 3.

TABLE 3

Viscosity
mPa * s

| solvent | E1 | E4* |
|---|---|---|
| glycerine | 87800 | 58200 |
| propylene glycol | 56200 | 43200 |

*comparative

It is observed that the Emulsion 1, according to the invention, has better thickening properties than the Emulsion of the prior art, both in water, and in glycols, and in emulsified water and oil systems.

1. An inverse emulsion comprising:
an aqueous phase, and
an organic phase
wherein:
the weight ratio of the aqueous phase to the organic phase is from 4:1 to 2:1;
the inverse emulsion includes from 20 to 70% by weight of an acrylic polymer;
the acrylic polymer is prepared by polymerizing:
i) 2-acrylamido-2-methylpropanesulfonic acid;
ii) at least one cationic acrylic monomer of formula I: 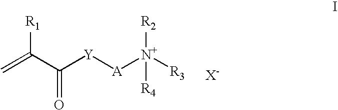
wherein R₁ is:
hydrogen or methyl;
R₂, R₃, R₄ are, one independently of the others, hydrogen or C₁-C₄ alkyl;
Y is NH or O;
A is a C₁-C₆ alkylene; and
X is an anion;
iii) acrylic or methacrylic acid; and
iv) at least one polyfunctional monomer; and
wherein from 1 molar % to 29 molar % of the total molar % of monomers i), ii) and iii) used to prepare the polymer is 2-acrylamido-2-methylpropanesulfonic acid.

2. The inverse emulsion of claim 1 wherein the cationic acrylic monomer of formula I is present in the acrylic polymer of the invention in a molar percentage of from 0.1 to 10% of the total molar percentage of monomers i), ii) and iii).

3. The inverse emulsion of claim 1 wherein the cationic acrylic monomer is acryloyloxyethyl-trimethylammonium chloride or methacryloyloxyethyl-trimethylammonium chloride.

4. The inverse emulsion of claim 1 wherein the polyfunctional monomer contains two or more unsaturated reactive groups and it is present in the acrylic polymer in an amount comprised between 0.01 and 1 mmoles per mole of monomers i), ii) and iii).

5. Inverse emulsion according of claim 1 wherein the polyfunctional monomer is methylenebisacrylamide.

6. The inverse emulsion of claim 1 wherein the organic phase is a component having boiling point from 150 to 300° C. selected from the group consisting of mineral oils containing saturated hydrocarbons; vegetable oils; and mixture thereof.

7. The inverse emulsion of claim 1 wherein the organic phase is selected from the group consisting of polydecene, isohexadecane, and a C₁₃-C₁₆ isoparaffin.

8. The inverse emulsion of claim 7 wherein the organic phase is a C₁₃-C₁₆ isoparaffin.

9. The inverse emulsion of claim 1 further comprising at least one emulsifier.

10. The inverse emulsion of claim 9 wherein the emulsifier is a water-in-oil emulsifier selected from the group consisting of sorbitan monolaurate, sorbitan monopalmitate, sorbitan monostearate, sorbitan monooleate, and mixtures thereof.

11. The inverse emulsion of claim 9 wherein the emulsifier is an oil-in-water emulsifier wherein the oil-in-water emulsifier is a linear or branched ethoxylated alcohol.

12. The inverse emulsion of claim 9 wherein the inverse emulsion includes both a water-in-oil emulsifier and an oil-in water emulsifier and a total quantity of emulsifiers of from 2 to 10% by weight and the weight ratio of water-in-oil emulsifier and oil-in-water emulsifier is from 2:1 to 1:2.

13. A method for the preparation of an inverse emulsion for cosmetic formulations comprising:
a. preparing a mixture comprising from 40 to 60% by weight of a water phase, and from 60 to 40% by weight of a mixture of acrylic monomers consisting of:
i) 2-acrylamido-2-methylpropanesulfonic acid;
ii) at least one cationic acrylic monomer of formula I: 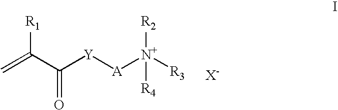
wherein R₁ is:
hydrogen or methyl;
R₂, R₃, R₄ are, one independently of the others, hydrogen or C₁-C₄ alkyl;
Y is NH or O;
A is a C₁-C₆ alkylene; and
X is an anion;
iii) acrylic or methacrylic acid; and
wherein from 1 molar % to 29 molar % of the total molar % of monomers i), ii) and iii) is 2-acrylamido-2-methylpropanesulfonic acid;
b. adding to the composition an alkali to regulate the pH between 4 and 7;
c. adding to the composition prepared in b from 0.1 to 10 mmoles of a polyfunctional monomer per mole of mixture of monomers i), ii) and iii) and an initiator of radical polymerisation while maintaining the temperature between 3 and 7° C.;
d. preparing an organic phase containing one or more water-in-oil emulsifiers;
e. introducing the mixture obtained in b. into the organic phase prepared in c. and emulsifying the two phases by stirring;
f. initiating the polymerisation and completing it while maintaining the temperature at from 55 to 95° C. under stirring; and
g. cooling the reaction mixture to 35-45° C. and adding thereto an oil-in-water emulsifier.

14. The method of claim 13 wherein 2-acrylamido-2-methylpropanesulfonic acid in the form of sodium salt is used and the pH is regulated in phase b using aqueous sodium hydroxide (NaOH).
